(19) United States
(12) Patent Application Publication
(10) Pub. No.: US 2009/0232899 A1
(43) Pub. Date: Sep. 17, 2009

(54) Mucoadhesive nanocomposite delivery system

(76) Inventors: Allan E. David, Ann Arbor, MI (US); Rulyun Zhang, York, PA (US); Yoon Jeong Park, Seoul (KR); Arthur Jin-Ming Yang, York, PA (US); Victor C. Yang, Ann Arbor, MI (US)

Correspondence Address: JONES DAY, 222 EAST 41ST ST, NEW YORK, NY, 10017, US

(21) Appl. No.: 11/596,934

(22) PCT Filed: May 20, 2005
(86) PCT No.: PCT/US05/17638

§ 371 Date: Aug. 29, 2007

Related U.S. Application Data
(60) Provisional application No. 60/572,953, filed on May 21, 2004.

(51) Int. Cl. A61K 9/50; A61K 38/43; A61K 38/00; A61K 31/165; A61K 31/43

(52) U.S. Cl. 424/501; 424/94.1; 514/2; 514/152; 514/197

(57) ABSTRACT

A nanocomposite delivery system uses chitosan as a mucoadhesive material encapsulated in a surface modified network of colloidal nanoporous nanoparticles, such as silica, or other colloid-forming materials, especially metal oxides. Drug delivery systems may be provided by binding a drug to the chitosan/silica nanocomposite, typically by adding a drug or other active agent during in-situ gellation of colloidal silica. When the active agent is, for example, amoxicillin or other antibiotic agent, the drug delivery system may be used in the treatment of stomach ulcers, for example.

This application claims the benefit of priority from U.S. Provisional Patent Application No. 60/572,953, filed May 21, 2004. The prior application, in its entirety, is incorporated herein by reference.

The invention relates to a drug delivery system that will adhere to stomach mucosurface. The invention also relates to a composite drug delivery system wherein a chitosan polymer is encapsulated with surface modified colloidal nanoparticles. The invention also relates to treatment of peptic ulcers caused by Helicobacter pylori (H pylori) by delivering a nanopore composite of chitosan biopolymer and a drug which is effective for treating H pylori in proximity to sites infected by H pylori.

Chitosan is a biocompatible and biodegradable material having the property of mucoadhesiveness and ability to sustain drug release.

In one embodiment, the present invention provides a composite drug delivery system made by encapsulating chitosan polymer with surface modified colloidal nanoparticles. According to this embodiment, in-situ gelation of surface modified silica particles, (such as disclosed in commonly assigned copending application Ser. No. 09/601,888, filed Aug. 9, 2000, and Ser. No. 10/110,270, filed Sep. 30, 2002, the entire disclosures of which are incorporated herein by reference), in the presence of chitosan, creates an interpenetrating network of silica and chitosan macromolecules. Silica gel, being very stable in acid, provides a tight entanglement structure in a silica-chitosan composite to significantly retard chitosan's leaching under acidic environment. Therefore, in the gastric environment, the composite is able to control the drug release rate more effectively than chitosan on a stand-alone basis.

In one embodiment of the invention an antibiotic drug, such as amoxicillin, is combined with a silica-chitosan nanocomposite as a delivery device, suitable for delivery in the gastric cavity.

In another embodiment of the invention, a polypeptide drug, such as EGF (epithelial growth factor), is combined with a silica-chitosan nanocomposite as a delivery device, suitable for delivery in the gastric cavity.

According to embodiments of the present invention, a tight silica pore structure surrounding chitosan is created which acts as a structural support. By controlling the pore structure of the silica-chitosan composite, the drug release kinetics may be moderated to maximize the efficacy benefits achievable by mucoadhesion.

The present invention, according to embodiments thereof, provides surface ligand groups incorporated onto the silica pore surface to enhance drug stability, diffusion, absorption, and permeation.

In one embodiment of the invention, decomposition of a peptide drug is reduced due to inhibition of membrane bound enzymes by the surface ligand groups.

In these embodiments, because of their close proximity, i.e., a few nanometers, to the entrapped drug molecules, the ligand groups provide a highly efficient local chemical environment that facilitates drug interactions.

Accordingly, in various embodiments of the invention, chitosan composites are provided for a variety of mucoadhesive drug delivery applications. The present invention, in its various aspects and embodiments, provides:
(1) an interpenetrating network of silica and chitosan that prevents chitosan's leaching into acid and controls drug release rates in the stomach;
(2) strong adhesion (by chitosan's cationic amino groups at acidic conditions) to the gastric mucosal surface that prolongs and enhances drug delivery near bacteria colonies;
(3) engineered pore structure (through morphology control at nanometer scale) that maximizes a drug's antibacterial performance by regulating its release rates;
(4) a dense layer of ligand groups on the pore surface of a nanoporous nanocomposite to facilitate drug delivery;
(5) an inhibition function, provided by surface ligands on a nanoporous nanocomposite, that retards the degradation of a (poly)peptide drug by deactivating a membrane enzyme at a site of adhesion, but not elsewhere;
(6) a core technology for the development of chitosan composites for additional mucoadhesive drug delivery applications.

### BRIEF DESCRIPTION OF THE DRAWINGS

The invention will now be described in greater detail with the assistance of specific, non-limiting embodiments thereof, and with the aid of the following drawings in which:

FIG. 1 is a schematic drawing of an embodiment of a silica-chitosan nanopore composite according to the present invention;

FIG. 2 is a schematic diagram broadly illustrating principles of the mechanism of the operation of mucoadhesion to the mucus layer of the stomach of a silica-chitosan composite material according to an embodiment of the present invention;

FIG. 3 is a schematic diagram broadly illustrating principles of the operation of target-specific adsorption using the composite material according to another embodiment of the present invention;

FIG. 4 is a graphical representation of the swelling ratio as a function of time of a chitosan-silica composite material according to an embodiment of the present invention;

FIG. 5 is a schematic illustration of the effects of aging and drying a wet gel precursor on shrinkage and pore structure of the dried composite material with and without the presence of surfactant support;

FIG. 6 is a schematic illustration of the swelling of embedded chitosan molecules in the composite material and corresponding reduction in pore size, according to an embodiment of the present invention;

FIG. 7 is a flow diagram illustrating steps which may be used to prepare a drug-delivery system in accordance with embodiments of the present invention;

FIG. 8 is a graphic drawing showing amoxicillin release profiles (accumulated concentration versus time) for two different air dried samples according to embodiments of the present invention;

FIG. 9 is a graphic drawing showing amoxicillin release profiles (percent released versus time) for three different composites, before (wet gel) and after drying (air dried or freeze dried), according to embodiments of the present invention;

FIG. 10 is a graphic drawing showing amoxicillin release profiles (percent released versus time) of another embodiment of a composite material according to the present invention, before (wet gel) or after (air dried or freeze dried) drying;

FIG. 11 is a graphic drawing showing the release profile (amount(%) released versus time) of metronidazole from a chitosan/silica composite material according to an embodiment of the present invention;

FIG. 12 is a graphic drawing showing the release profile (amount(%) released versus time) of metronidazole for different molecular weight chitosan: Low molecular weight (LMW); Medium molecular weight (MMW); or High molecular weight (EMW) chitosan; and

FIG. 13 is a schematic illustration showing the preparation of a spherical composite material by an emulsion process according to one embodiment of the present invention.

The following table illustrates differences between various parts of the gastrointestinal (GI) tract when comparing several key factors that impact general GI drug delivery.

| | |
|---|---|
| Stomach | Small |

| | Fasting | Food | Intestine | Colon |
|---|---|---|---|---|
| Retention | 2 hr | 3-6 hr | 2-4 hr | >12 hr |
| pH | 2 | 5-6 | 6-7 | 6-8 |

| | | | |
|---|---|---|---|
| Surface area | ~3 m² | ~400 m² | ~1 m² |

To account for these differences, the moderation of a drug's absorption may be at least as important as, if not more important than, the control of its release rate. Furthermore, many other factors such as degradation by enzymes, undesirable side effects and bioavailability can be taken into consideration in the overall design of a delivery mechanism intended to enhance efficacy. For drugs that are susceptible to degradation by intestinal enzymes or inactivation by drug transporters (e.g., p-glycoprotein efflux system), complete absorption in the upper GI tract would be beneficial. For many antibiotics, the optimal absorption point is the stomach or upper intestine because of concerns over altering the normal flora of the GI tract, particularly the flora of the colon. The bioavailable dosage of a drug, represented either by the area under the plasma concentration-time curve, or the sustaining local concentration at a target site, should be sufficiently high to achieve the effectiveness of the drug for its intended purpose.

In the upper GI tract, the short duration (less than 4˜6 hours) of a drug dosage often hinders its absorption there. Simply controlling the release rate may not be adequate for delivering a drug within such a narrow absorption window. To extend the drug's residence time in the GI tract various and known expedients can be used, such as, for example, (a) a low-density dosage form that floats above gastric fluid; (b) a high-density form that is retained in the bottom of the stomach; (c) an expandable (by swelling) form that restricts emptying through the pyloric sphincter; (d) a muco-adhesive form that adheres to stomach mucosa; or (e) a concomitant administration of drugs that slows the motility of the GI tract.

The sustained release of a drug in the stomach should benefit the treatment of stomach illnesses, such as, peptic ulcers. In 1982, Australian physicians Robin Warren and Barry Marshall first identified the link between H. pylori and ulcers, which led to the conclusion that this particular bacterium, not stress or diet, caused ulcers. In 1994, a National Institutes of Health Consensus Development Conference confirmed the linkage between H. pylori and ulcer disease, and recommended that ulcer patients with H. pylori infection be treated with antibiotics.

The present inventors hereby describe the synthesis of silica-based nanocomposites with precisely controlled composition, morphology, and particle size providing a high loading of surface ligands (e.g., up to about 4 to 5 ligands/nm² or up to about 7.5 mmole ligand per gram of silica gel). Surface modified silica based nanogels are described in the aforementioned commonly assigned co-pending applications U.S. Ser. No. 09/601,888 (see also WO 99/39816, re-published Apr. 24, 2000) and U.S. Ser. No. 10/110,270 (see also WO 01/17648, published Mar. 15, 2001). Such surface modified silica gel may be referred to herein as “CSMG.” The inventors also describe embedding reactive substrates within the surface modified nanopore structure to integrate the adsorption, reaction, and catalysis functions into one operation.

According to an embodiment of the present invention, a mucoadhesive chitosan nanocomposite that substantially elevates drug efficacies in treating peptic ulcers is synthesized from colloidal silica and chitosan. This nanocomposite possesses several unique properties that make it highly suitable for upper GI tract delivery.

Chitosan is a polysaccharide derived by deacetylation of chitin, an abundant by-product of shellfish.

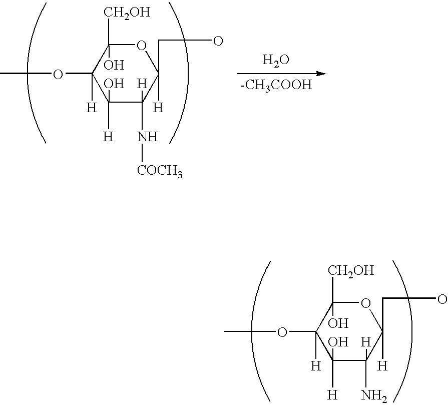

Chitosan is biocompatible and biodegradable and additionally demonstrates one or more favorable characteristics, such as mucoadhesiveness, permeation enhancement, and sustained release. Chitosan is characterized by its high solubility in strong acid, such as in the environment of the stomach.

CSMG is an organic-inorganic nanopore composite with an exceptionally large surface area (˜900 m²/g) and high ligand loading (4˜5 ligands/nm²). The open channel structure allows full access to the embedded entities. The key to producing a nanocomposite with an open channel structure (and high surface area) is to maintain phase compatibility at the nanometer scale.

Preventing sub-micron phase separations may be achieved by controlled processing which encompasses balancing the amount of co-solvent, surfactant, and co-surfactant and precisely controlling the ionic strength.

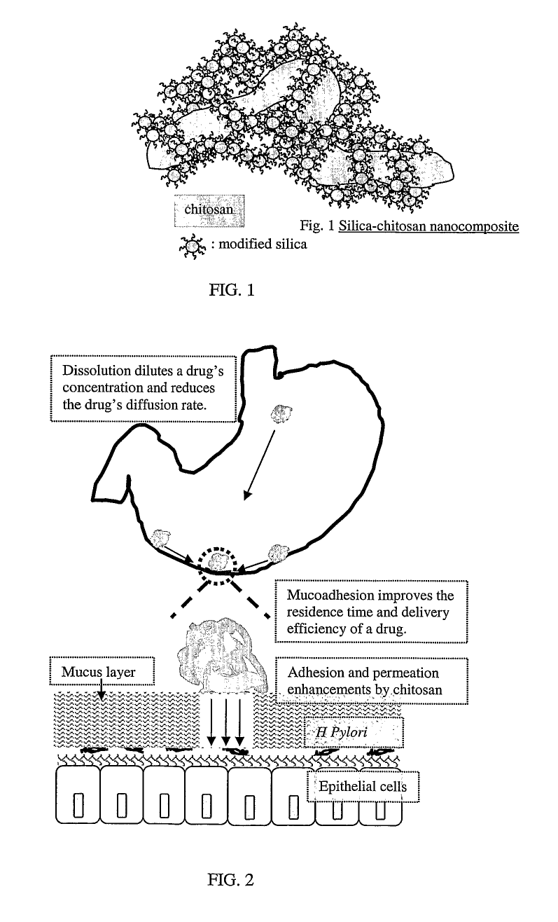

According to embodiments of the present invention synthesis of a silica-chitosan nanopore composite is achieved by dissolving the chitosan polymer into acidic, surface modified, silica sols and then inducing an in-situ gelation by raising the pH to a neutral pH, such as about 7. The in-situ gelation of colloidal silica nanoparticles with the presence of a dissolved polymer creates an interpenetrating network (IPN) between silica and chitosan. The silica-chitosan nanopore composite as shown schematically in FIG. 1 has several attributes that are ideally suited for drug delivery in the stomach.

Strong silica-chitosan charge interactions and the swelling of chitosan polymers by acid can be used to manipulate pore morphology during gelation. Coupling this morphology control with various schemes of silica drying (e.g., ambient, supercritical, or freeze drying) and aging (acidic, basic, or controlled ionic strength) provide numerous options for material optimization, including composite mechanical strength and drug diffusion rate. Furthermore, the abundance of surface ligands on the silica particles allows the microenvironment (pH, ionic density, hydrogen bonding, polarity, drug affinity) surrounding a drug at or near the delivery site to be moderated.

The use of a silica-chitosan composite for drug delivery according to embodiments of the present invention may provide one or more of the following features.

Due to adhesion to the mucosal surface by chitosan, this composite should (1) prolong the drug's residence time in the stomach; (2) increase the drug concentration gradient across a membrane; and (3) enhance drug permeation (most likely through the opening of the intercellular junctions) into a membrane.

The substantial protonation of chitosan's amino groups should (4) decelerate the decomposition of antibiotics by infused gastric acid.

The nanopore silica structure tightly surrounding a chitosan polymer should (5) prevent the leaching of chitosan (and the burst release of drugs) in the harsh acidic environment and (6) allow reversible chitosan composite swelling with changes of pH.

The silica pore structure and ligand groups should be useful to (7) further modify the drug release rate, and (8) provide a local chemical environment around a delivery site for additional enhancements in drug permeation, absorption and stability.

FIG. 2 shows some of these effects in a stomach environment.

According to one embodiment of the invention, the noninvasive delivery of (poly)peptide drugs, various enzyme inhibitors, chemically bonded to amino groups of chitosan, may be used for prevention of peptide decomposition by membrane enzymes. Enzyme inhibitions by surface ligands on silica should leave more amino groups of chitosan for adhesion. Because of enhanced mucoadhesion, the enzyme inhibition by the surface ligands on silica would be close to a cell surface, near the drug delivery site and confined locally. Therefore, it would be more effective in protecting against degradation, yet less harmful to the body's general metabolism.

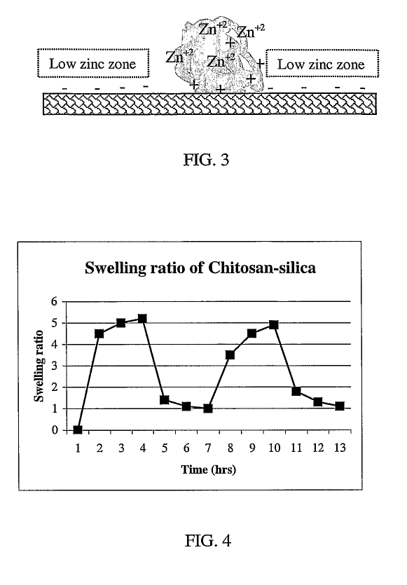

In another embodiment of the invention, molecular recognition ligand groups are incorporated to achieve a high-efficiency, target-specific adsorption. FIG. 3 illustrates the depletion of zinc ions using ethylenediamine (a chelating agent for zinc ion) modified silica to create a zinc-deficient local environment around a peptide drug, thereby reducing its decomposition by membrane bound metallo-peptidases.

In the silica-chitosan delivery system, drug loading occurs through embedding during the gelation process, rather than via adsorption afterward. This loading method allows for improved control of the drug release rate. At present, many mesoporous drug carriers are prepared by partitioning (i.e., absorbing a drug from its solution). Although a nanopore substrate has a large surface area, the loading capacity might still be limited by diffusion kinetics and polarity differences. In addition, loading by diffusion may not be practical for a sustained release device where the diffusion rate is inherently low.

Dissolving a drug before gelation enables it to be uniformly distributed within the composite medium before pore formation. This is an ideal method of filling drugs into the smaller nanopores which are less accessible by diffusion. This uniformity is extremely important in achieving a consistent product quality. Compatibilizing organic and inorganic components allows this process to be effectively and efficiently performed. Phase compatibilization at submicron length scales is an important element in producing a uniform nanocomposite which allows uniform loading of both hydrophilic and hydrophobic drugs into the nanocomposite.

Thus, in one embodiment, the present invention provides a silica-chitosan nanocomposite that is mucoadhesive, stable in acidic environments and retained longer in the stomach and which can be synthesized using a sol-gel process such as previously described for the production of CSMG.

A nanocomposite according to this embodiment of the invention may be produced with a controlled pore morphology by design and selection of composition ratio (e.g., amount of initial solvent) and processing conditions (e.g., pH control, aging and drying of silica). Controlling pore morphology may be used, for example, to better fine-tune drug release from the composite, such as, in response to a low pH.

In a specific embodiment, the present invention provides a mucoadhesive nanocomposite drug delivery system which incorporates one or more drugs effective in the treatment of ulcers.

In a related aspect, the present invention provides a method for treating stomach ulcers by administering to a patient in need of such treatment a mucoadhesive nanocomposite drug delivery system comprising a pharmacologically effective amount of a drug effective for the treatment of stomach ulcers.

In another embodiment, the present invention provides a silica-chitosan nanocomposite which includes a pharmacologically effective amount of a drug which has been loaded into the composite by entrapment before gelation. Representative drugs which may be embedded according to this embodiment of the invention include, without limitation amoxicillin and/or other antibiotic agents effective in the treatment of stomach ulcers and epidermal growth factor (EGF).

In an embodiment of the invention, initial work with the nanocomposite drug delivery system will be used to obtain concentration versus time profiles from simulated drug release experiments and these profiles may be used to correlate the silica pore structure. By optimization of the silica pore structure combined with pharmacokinetic and pharmacodynamic studies, it will be possible to determine the best mode of delivery performance.

While the above description refers most generally to silica based nanocomposites, chitosan nanocomposites according to various embodiments of the invention may be produced using similar procedures which a wide range of colloidal materials. As examples of such colloid-forming materials mention may be made of, for example, metal oxides, such as, alumina, zirconia, titania, and the like, including mixtures of metal oxides. As well known in the art, gels of the metal oxides may be prepared similarly to the silica gels, such as, for example, from the corresponding metal hydroxide precursors.

The particular colloidal material may be selected depending on, for example, the particular application for the mucoadhesive chitosan-based nanocomposite drug delivery system. For example, mention may be made of novel composites effective for the non-invasive administration of therapeutic peptides by, for example, nasal sprays, implants, and other delivery methods that require blood compatibility.

### Silica-Chitosan Nanocomposite

A silica-chitosan nanocomposite is made from 3 ml of silicic acid obtained from an ion-exchange process (see experimental design section) and 1.5 ml 2% chitosan solution. The sample is gelled, aged for one day and ambient dried. The composite shows reversible swelling in response to pH changes. The following are results of swelling tests.

| | Time (hr.) | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Ph | 5 | | | 8 | | | 5 | | | 8 | | |
| Swelling | 4.5 | 5 | 5.2 | 1.4 | 1.1 | 1.0 | 3.5 | 4.5 | 4.9 | 1.8 | 1.3 | 1.1 |
| σ (deviation) | 0.25 | 0.3 | 0.3 | 0.27 | 0.1 | 0.09 | 0.6 | 0.4 | 0.3 | 0.08 | 0.3 | 0.2 |

The results (also shown graphically in FIG. 4) demonstrate that the silica nanopore structure prevent chitosan from leaching in acidic environments. It is evident that this composite is capable of both moderating drug release and protecting the drug from degradation.

### Loading Reactants by In-Situ Gelation

One major benefit using sol-gel chemistry is that a drug can be loaded into a composite by in-situ gelation of silica. Loading a drug by entrapment via gelation provides highly uniform drug distribution and allows for the independent control of release rates through pore structure manipulations. In the development of CSMG, enzyme, reactant (e.g., iron) particles, and oil phases were embedded respectively within silica nanocomposite via gelation. In an embodiment of the present invention, oil is embedded in the silica-chitosan nanocomposite to not only reduce pore shrinkage, but also to dissolve a hydrophobic drug, prevent drug decomposition by acid prior to gelation, and moderate the drug diffusion rate.

### Controlling Pore Morphology

In an embodiment of the invention, drug release rates may be fine tuned by performing surfactant templating during processing followed by shrinkage control during drying and aging. The present invention also includes, in various embodiments thereof, morphology preservation through the application of (i) different drying (i.e., supercritical, freeze, and ambient) schemes, and/or (ii) a hydrolyzed silane coupling reagent (as a reactive surfactant) to simultaneously stabilize a microemulsion and modify the silica pore surface.

### Experimental Design and Methods

In 1992, scientists at Mobil developed a family of mesoporous molecular sieves (M41S) [see, e.g., J. S. Beck, et al., J. Am. Chem. Soc. 114 (1992) 10384; C. T. Kresge, et al., Nature 359 (1992) 710) using cationic surfactants to assemble silicate anions from solution. The micellar assemblies of quaternary ammonium cations served as the structure-directing agents. Their strong electrostatic interactions with anionic silicate oligomers led to condensation of inorganic precursors around the regular structure, forming a continuous silica phase with templated pore morphology. Three different pore geometries were made: MCM-41 (hexagonal), MCM-48 (cubic), and MCM-50 (laminar).

According to an embodiment of the present invention, a silica-chitosan composite is produced by a process incorporating templated pore morphology which is based on chitosan being a cationic polymer. Instead of self-assembling into a regular micelle structure like quaternary ammonium surfactants, chitosan maintains a polymeric conformation determined by the solution environment (mostly by pH). With the presence of anionic silica, the strong charge interactions lead to a stable structure in solution that, after the gelation of silica nanoparticles, turns into an interpenetrated network as shown in FIG. 1. The strong interaction between polyelectrolytes may stabilize microphase domains and preserve morphology while the composite is undergoing aging and drying. In addition, the strong interaction may be further utilized for engineering composite pore structures. These procedures should provide the desired material characteristics at the nanoscale level.

The following section details series of experiments designed to (1) establish the correlation between composite properties, particularly mechanical strength, pore morphology, and surface ligand density, to several key processing steps in composite synthesis; (2) establish the correlation between the control of release rate to the structure and composition details and, subsequently, the synthesis conditions of the nanocomposite (amoxicillin is used as a model drug for the study of controlled release under simulated gastric environments); (3) utilize animal models to demonstrate the effectiveness in gastric retention as well as the in-vivo efficacies of antibiotics and EGF delivered by a silica-chitosan composite; and (4) achieve material characterizations to understand details of the nanostructure and its relation to synthesis conditions.

As will be discussed in detail later, an aspect relevant to the series of experiments (1) is to control the condensation of the surface Si—OH groups that still remain after gelation.

Data from the series of experiments (2) may be used for additional modifications in composite synthesis to control the composite's composition and morphology so that the drug is released over a time period longer than twelve hours and for the release kinetics to approach zero order kinetics.

Chitosan-Based Nanopore Composite from Colloidal Particles

During gelation and surface modification, the interfacial properties are finely tuned so that the prescribed morphology is stabilized either kinetically or thermodynamically. For the synthesis of CSMG, the surfactants of choice simultaneously achieve several tasks, namely achieving compatibility between reaction systems, creating morphology through self-assembly, supporting pore structure against shrinkage, preventing crosslinking of surface silanols, and facilitating the surface modification reaction of silica. Because of the large interfacial area inherent in a nanocomposite system, the choice of solvents and surfactants is precisely balanced to avoid the need for excessive surface compatibilizers. According to embodiments of the present invention, a system is utilized in which a reactant or reactants also function as surfactants. For example, a silane coupling agent may be used simultaneously as a surface ligand reactant as well as a phase compatibilizer in the same system.

The Manufacture of a Nanopore Composite from a Sol-Gel Reaction of Modified Colloidal Silica

Silica sols exist as stable colloidal nanoparticles in water. These particles can be easily precipitated out or gelled by a change in pH, such sol-gel reaction producing a nanopore substrate. The source of silica sol can be, for example, alkylated silicates or sodium silicates. Silica sol can be obtained from the hydrolysis of TEOS (Si(OC₂H₅)₄) or TMOS (Si(OCH₃)₄). Silica sol-gel chemistry may be described by the following reaction schemes:

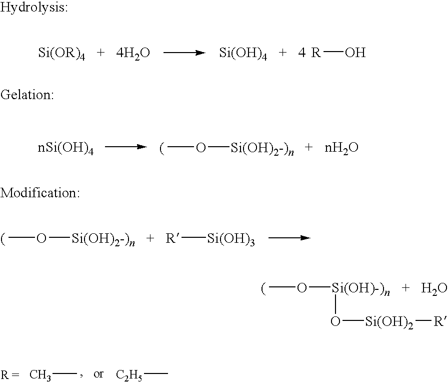

Silicic acid, (Si(OH)₄), may be obtained through an ion-exchange process directly from sodium silicate without the generation of any alcohol by-products (see reaction scheme below):

Ion-exchange: Na₂SiO₃+H₂O+2H⁺-Resin→Si(OH)₄+2Na⁺-Resin

This method is extremely suitable for encapsulating drugs or biological entities, or any other compounds or materials having low tolerances to alcohol.

The silicic acid process also has the advantage of a much lower ionic strength when compared with sol-gel processes that use colloidal silica instead of TEOS or TMOS. The low charge content may be critical for the inclusion of hydrophobic components (such as a surface modifying agent or drug) because of the improved solubility of organic components at low ionic strength. Freshly prepared silicic acid (silica sol) is composed of silica particles with a size of several nanometers, so the gelled structure has a large surface area with many active silanol groups. When gelation occurs, the silica sols quickly form a Cayley tree branching structure. The formation of the backbone chain bonds will only consume two of the four silanol groups of each monomer. After gelation, half of the silanol groups are still unreacted.

The reactive silanol groups can be used for the incorporation of surface modifying groups. If unreacted with other functional groups, surface silanol groups will react with each other to form ring-closing bonds, resulting in gel crosslinking and pore shrinkage. Ring bond formation occurs naturally during aging of silica gels and yields gels with greater mechanical strength.

The present invention may take advantage of manipulation of surface Si—OH groups during different stages of composite processing. At a pH above silica's isoelectric point (pH˜2), these groups are negatively charged. Their presence stabilizes the chitosan, which is cationic. During aging and drying, these Si—OH groups react with each other in a condensation reaction which strengthens the backbone structure of silica. The aging is necessary not just for gaining gel strength, but also for reducing the negative charge density (of Si—OH) on the silica surface. A highly negative surface charge density could compromise the mucoadhesiveness of the chitosan. These silanol groups may be reacted with a coupling reagent to incorporate ligand groups with special functionality.

To incorporate a ligand group R′, we normally start with a silane coupling agent of the formula R′—Si(O C₂H₅)₃. Because of their frequent use in electronics-related applications, a host of silane coupling reagents are available commercially (see, e.g., www.gelest.com) that can be used for surface modifications (for example, R′═—CH₂CH₂CH₂SH, —CH₂CH₂CH₂NH₂, —CH₂CH₂—O—CH₂OH, —CH₂CH₂CH═CH—COOH, etc.).

After hydrolysis, the silicon end becomes Si—OH, which is identical to silica sol, meaning that the silane coupling reagent could also function as an extra surfactant in stabilizing the mixture of silica sol and other organic components. Consequently, synthesis of the silica-chitosan composite may begin with a batch of modified silica sol made by reacting silicic acid with a coupling reagent.

The following is a typical example of silica sol modification with mercapto (—SH) ligand groups. Silica sol is obtained either from sodium silicate by an ion-exchange process or from the preparation of TEOS, H₂O, ethanol and HCl, in the total molar ratio 1:2:4:0.0007. The mixture of 50 ml of silica sol and a variable amount (depending on the desired % of ligand loading) of 3-mercaptopropyltrimethoxysilane are added into a reaction vessel equipped with an agitator, heating mantel, thermometer and nitrogen purge system. Additional water or ethanol is used to adjust the solvent/co-solvent ratio in the solvent mixture so that their proportions are suitable for the amount of ligand desired. The reaction mixture is heated to 50-60° C. for 1˜2 hours and then cooled to room temperature.

To make a silica-chitosan composite from this mixture, the modified silica is mixed with 25 ml of 2% chitosan in acetic acid and then gelled by an adjustment of pH with NH₄OH (or NaOH) solution. The loading of drugs and/or other formulation additives are done prior to gelation. Depending on the detailed chemistry required, other co-solvents or surfactants may be added to improve phase compatibility. Sub-Micron phase homogeneity may be measured using Dynamic Light Scattering (DLS). After gelation, the composite is subjected to designed aging and drying sequences in order for the pore morphology to be controlled properly.

### Morphology Creation and Preservation During Processing

The pore morphology of the composite is controlled to effect the desired drug release rate of that composite.

Prevention or minimization of porosity loss due to shrinkage may be accomplished by the procedures described herein. Shrinkage during drying is induced by the capillary stress of interfaces, particularly from the water-air meniscus. The following conjecture shows that capillary stress is inversely proportional to pore size.

$$\begin{matrix}Capillary stress = \frac{surface force}{surface area} \\ \approx \frac{σ \cdot 2 π r}{4 π {r}^{2}} \\ = \frac{σ}{2 r}\end{matrix}$$

where σ=surface tension and r=radius.

For nanometer size pores, the stress can be in the range of 100 Mega Pascal (MPa). The smaller the pore size, the bigger is the stress concentration. This type of force will collapse the pore if the supporting fluid contains a vapor phase (infinite compressibility) or if the strut density is low. Even if the pore structure does not buckle under the stress, condensation of surface Si—OH across a pore would make the shrinkage effect permanent. The loss of porosity would be more prominent in smaller pores. This permanent shrinkage would close off some channels and block drug permeation. Consequently, shrinkage during drying needs to be controlled carefully.

Ambient Drying with Support

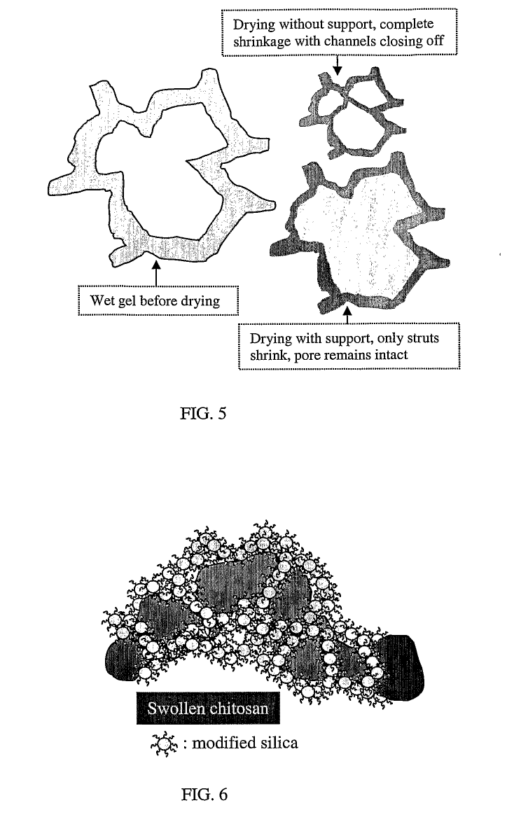

Previously developed technology provides a synthesis process for creating and maintaining a high amount of mesopores with minimum shrinkage. This procedure utilizes surfactant self-assembly to create a desired morphology and then relies on the same surfactants to support the pore structure against shrinkage during aging and drying. This procedure along with adequate aging, promotes condensation of silanol groups primarily in the strut, not across pores, thereby restricting shrinkage primarily to the strut areas, making the backbones stronger, while leaving the vast pore volume unaffected by drying. This phenomenon is illustrated in FIG. 5.

According to embodiments of the present invention, the cationic chitosan polymer will support the pore structure even better than the cationic surfactants would because of its high viscosity. Although chitosan cannot self-assemble into a regularly shaped domain due to its high molecular weight and viscosity, it has several other advantages over cationic surfactants. Its drying rate and swelling ratio can be adjusted over a fairly wide range by a simple change in environmental pH. This unique attribute can be exploited to facilitate morphology control of the silica-chitosan composite. Air-dried samples may be prepared by allowing solvent evaporation at 37° C. for an adequate time.

### Freeze Drying

The silica-chitosan composite may be freeze-dried by rapidly freezing at, for example, about −80° C. and dried in a freeze-dryer. The freeze-dried sample is expected to preserve the initial pore volume because there would be much less shrinkage resulting from a low surface stress (no liquid-vapor meniscus). However, its mechanical strength may be weak due to the lack of crosslinking, normally enhanced by an ambient drying, within the strut.

Optimization and comparison between alternative aging and drying methods may be determined. For example, the following types of procedures may be carried out:
(1) silica-chitosan composites are aged under three pH levels: 4, 7 and 9, for one to two days;
(2) aged samples are dried under two different conditions: ambient, freeze-drying; and
(3) samples obtained from different aging and drying conditions are characterized for physical dimension, pore volume, and surface area (as well as mechanical modulus, if necessary).
   The data regarding pore volume and surface area should be sufficient to verify whether the morphology control is effective. More complete pore characteristics may, if desired, be obtained, for example, chitosan can be removed after drying by calcination (to 630° C. for 4 hours with a heating rate at 2° C./min).

### Loading Drug in Silica-Chitosan Composite

In order to load active agent such as drug or other pharmacological agent, the silica-chitosan composite may be made by an in-situ gelation of colloidal silica in the presence of chitosan polymers. The formation of an interpenetrating network between two polymers would be utilized to improve various properties of the composite ranging from mechanical strength to chemical stability. Additionally, the numerous surface hydroxyl groups of silica can be modified with ligand groups to moderate the chemical environment near a site of delivery.

In order to achieve drug loading by entrapment, an adequate amount of the desired drug and its accompanying formulation additives are mixed uniformly with chitosan and the colloidal particles prior to gelation. Otherwise, a premature phase separation, even one at micron scale, might later affect the drug release rate adversely. This problem could be induced by any one of several unforeseen interactions among the ingredients, including insufficient solvation, hydrophobic bonding, Coulomb interactions and high interfacial energy.

The following describes one representative example of embedding an active agent in a silica-chitosan composite according to embodiments of the invention. Similar procedures may be adopted using a different colloidal system in place of colloidal silica.

A mixture of 50 ml stripped silicic acid solution and an appropriate amount (10˜25 ml) of 2% chitosan (M˜70,000) in 2% acetic acid is stirred at room temperature for ten minutes as a starting solution. For including a hydrophilic ingredient with high solubility in water, an aqueous solution of the ingredient is mixed directly with the stock solution followed by gelation with the addition of a base.

For an embedded ingredient susceptible to acidic conditions, a quick mixing followed by immediate neutralization and gelation can minimize its exposure to acid. Mixing it with an oil phase prior to addition can further protect such an ingredient. During processing for CSMG material, a significant amount of an oil phase, e.g., vegetable oil, may be added to support the silica pore structure against shrinkage. Adding an oil phase to the accompanying surfactants allows for incorporating a sufficient amount of hydrophobic ingredients. The quality of mixing can be monitored with, for example, Dynamic Light Scattering (DLS) equipment. DLS is capable of measuring drop sizes ranging from a few nanometers to one micron.

In order to stabilize a water-oil microemulsion, in addition to regular surfactants, a co-surfactant, such as a hydrolyzed silane coupling reagent (R—Si(OH)₃), may be used. A silane coupling agent, after hydrolysis, contains the silanol groups on one end and an attached functional (R, organic) group on the other end. In a state of microemulsion, the silanol end can penetrate the silica sol phase while the remaining molecular chain (R) stays within the oil phase. This configuration is ideal for ligand incorporation, while also maintaining the morphology created by the water-oil microemulsion. In this case, the hydrolyzed coupling agent functions as an additional surfactant to stabilize the morphology. When the gelation reaction occurs, the silanol groups of the coupling agent, with its organic portion stuck in the hydrophobic phase, should react only with silanol groups near the water-oil interface, completing the designated surface modification. The aging process that follows would be more controllable because all of the interfacial silanol groups have already reacted with the coupling agent and would no longer take part in crosslinking reactions across the pore.

An example of the procedure mentioned above may include mixing an adequate amount of olive oil (or corn oil, etc.) with a hydrophobic ingredient (e.g., a polypeptide drug such as EGF) and a surfactant (for example, polyoxyl 40 hydrogenated castor oil NF, Cremophor RH 40, emulsifying agent, HLB 14-16). This mixture is then added into a silicic acid/chitosan stock solution along with a sufficient amount of pre-hydrolyzed silane coupling (R—Si(OH)₃) reagent as cosurfactant to create a microemulsion, which protects the drug from the acidic chitosan solution. The oil phase of the drug, with the help of surfactants, will be finely dispersed in the chitosan solution. The choice of the R group should be based on both the need for phase compatibility as well as the control of diffusion for drug release. The mixture is reacted at room temperature for ½ hour before inducing a gelation with the adjustment of pH. The optimal mixing and gelation conditions (i.e., temperature, pH change rate, ionic strength) to achieve both the uniformity of the ingredient distribution and the mechanical strength of the composite for the intended drug delivery task may be determined for each particular system by routine experimentation.

Once the drug loading procedure is completed, the stability (activity) of the loaded drugs, e.g., Amoxicillin and EGF, may be examined, for example, by in vitro antibiotic activity test and ELISA method, to confirm that the loading procedure does not affect the activity of the drugs.

The mucoadhesion and permeation enhancement are, at least in part, believed to be the result of cationic charges on chitosan. Although silica pore structure is quite open, a large amount of silica in composite may still hinder the effects of chitosan's charges. In order to address this potential situation, amino ligands may be incorporated on the silica pore surface to further increase cationic charge density. An alternative drug having a relatively high pKa compared to chitosan, e.g., tetracycline antibiotic, could also be used. The average zeta potential of chitosan microspheres is increased from +7.45 to 26.68 mV after loading with tetracycline whose amino groups have a pKa of 9.69 compared to 6.3 of chitosan. Similar practices could be used to increase the cationic charge density of a chitosan composite for a stronger mucoadhesion and permeation enhancement.

Embodiments of the present invention provide procedures for modifying drug release rates. In an interpenetrating network, the embedded chitosan polymers are tightly surrounded by many silica nanoparticles, yet are connected to each other through an open-channel pore structure. This microstructure could be further engineered by processing to control the release rate of an entrapped drug. The gelation and subsequent aging of silica will solidify a permanent pore structure. Morphological changes in the chitosan polymer in response to pH changes will be used to control the pore structure and moderate the release rate of the composite.

Effects of processing and material composition on the pore morphology are described below.

The effective diffusion rate of a drug out of a composite structure is determined by several factors. The most influential is the amount of porosity after aging and drying. The morphology of the pores and channels dictate the tortuosity and length of a diffusion path. The silica backbone structure does not affect the diffusion much except by defining the pore morphology. The surface ligand groups do affect the transport rate according to their affinity to a diffusant. Because of the large surface area, this retention by adsorption, as observed in the CSMG product, could be appreciable.

These effects may be examined with a series of designed experiments. The porosity of the composite is normally controlled by the amount of an evaporable solvent (and co-solvent) used in processing. During drying and aging, the porosity and channel structure will change due to shrinkage caused by surface stress and subsequent crosslinking.

Similar to previously developed processes, the initial structure of the silicate will be determined by the chitosan polymer's morphology because of the strong anionic-cationic interaction. Thus, initial pore morphology will be largely influenced by the pH of the solution prior to gelation (the processing pH). The pH is adjusted to neutral for gelation. During aging, the morphology of chitosan may still change, but at a slower rate (restricted by the gelled structure) in response to silanol crosslinking or additional pH (aging pH) change. When drying, the chitosan phase will lose water and shrink, creating voids in the composite. How much of the void volume will remain after a complete drying depends on the choice of drying method. If an oil phase is initially added, it is likely to stay primarily within the void volume after drying is complete.

An oil-containing CSMG sample with very low shrinkage when dried in ambient conditions has been prepared. The water and oil phases, with the help of surfactants and cosolvent, form a stable microemulsion which later support the pore structure against shrinkage. Furthermore, the addition of oil phase and surfactant significantly reduce the interfacial tension, which is the main driving force to collapse pores. Further modifications include utilizing an oil phase to incorporate a hydrophobic ingredient with the chitosan (water) phase. Additionally, moderating the amount of oil phase will facilitate control of the overall diffusion rate through the composite. The solubility of a drug in the oil phase must also be taken into account when evaluating the drug release rate. For drugs not soluble in the oil phase, either suspension or emulsification of the drug in the oil phase may be achieved by using an appropriate surfactant. The introduction of an oil phase in the preparation step may facilitate maintaining the drug's stability since it avoids direct contact between acidic water and the loaded drug.

The following description focuses on delivering antibiotics in a gastric environment. The acidic swelling of chitosan and interlocking structure of silica would dictate the overall drug release rates of a composite in that environment. At an acidic pH, the chitosan molecules swell substantially because of the electrostatic repulsion of the positively charged amino groups. The chitosan phase will swell in the stomach's acidic environment. The void will be gradually filled by the expanding chitosan and permeated water. FIG. 6 illustrates that pore volume, of the silica-chitosan nanocomposite, with chitosan in swollen state, is reduced because of the swelling of chitosan at a low pH (compared with FIG. 1).

After swelling, the embedded drug has three possible paths to diffuse out; (a) through the swollen chitosan, (b) through the water phase in a void, or (c) through the oil phase in a void. Because of the volume increase from swelling, the diffusion time through chitosan will be longer, achieving a sustained release.

With the restriction of the silica structure, the whole composite will swell reversibly with a change of pH. Control of this phenomenon may be used to further optimize the rate of drug delivery. For example, the substantial swelling of chitosan under a low pH may be used as a factor for controlling the effective diffusion rate within the channel structure. By experimenting with different composition ratios of silica and chitosan, the release rate under a gastric environment may be optimized. One strategy is to learn how to utilize the kinetics of swelling, the amount of the oil phase, and the pore morphology to fine-tune the release rate of the drug.

For example, amoxicillin release may be characterized under different pH values. The reversible swelling ratio of silica-chitosan composites at various pH levels may be determined separately by concurrent experiments.

From these analyses, the release rate of a drug may be adjusted by varying composition and processing conditions, such as (a) initial pore volume (determined by the solvent amount), (b) silica to chitosan ratio, (c) amount of oil phase, (d) processing pH, (e) aging pH, and (f) drying methods (ambient, or freeze drying).

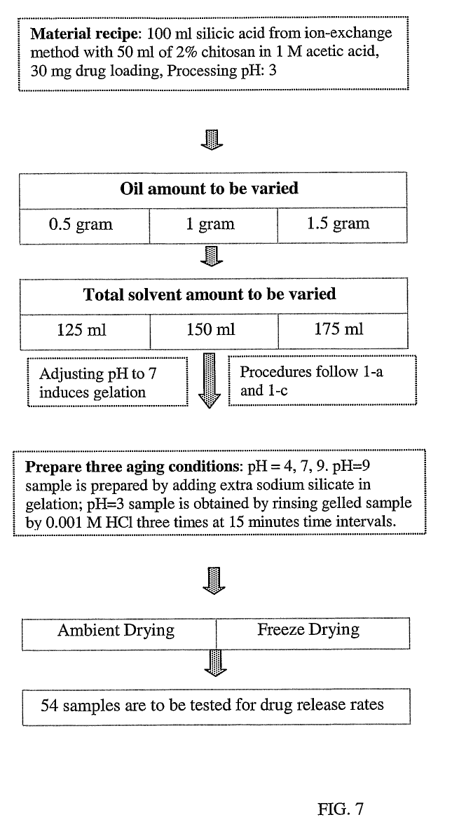

With this variety of options for changing the diffusion rate, the pore structure may be adjusted for the purposes of fine-tuning the drug release rate. However, the complexity of these interacting processing parameters requires a thorough study using a pre-designed experimental process. Based on preliminary experimental data on this system, the recipes and procedures shown in FIG. 7 have been designed.

Drug release rate may be assayed according to the following procedure.

Amoxicillin will be entrapped as a model drug according to the procedures described previously. 100 mg of amoxicillin-loaded silica-chitosan composite is incubated with 10 ml of simulated gastric fluid (prepared according to protocol described in US pharmacopeia) at 37° C. Its release rate will be established by assaying its concentration (i.e., adsorption at 276 nm) using a spectrophotometer (for example, Hitachi U-32 10, Japan).

A typical release curve should reflect at least two characteristic time constants: one for chitosan swelling (i.e., diffusion of water molecules), and one for diffusion of drug molecules. The data will be analyzed along with data from a swelling test.

Although several antibiotics such as ampicillin, gentamycin and tetracycline are effective against H. pylori in culture, their clinical use in ordinary dosages has not been effective in eradication of this organism. To be clinically effective, the antibiotics must penetrate through the gastric mucus layer and maintain a sufficiently high concentration (for antibacterial activity) near the infected site over a long period of time.

These requirements are achieved by a silica-chitosan composite according to embodiments of the present invention because of its swelling at low pH and its adhesion to the gastric mucosal surface. The following example demonstrates this effectiveness of the silica-chitosan composite.

Amoxicillin-loaded silica-chitosan nanocomposite is orally administered to 7-week-old male specific-pathogen-free Mongolian gerbils. The amoxicillin dose is adjusted to 10, 20, 30 mg/kg of body weight (3 groups). A group administered with 20 mg/kg standard amoxicillin suspension serves as the control group. The amoxicillin-loaded carrier is administered as follows: the amoxicillin-loaded nanocomposite is placed in a polyethylene tube (Intramedic Polyethylene Tubing; inner diameter, 1.14 mm; outer diameter, 1.57 mm; Becton Dickinson and Company, Sparks, Md.), one end of which is covered with hydroxypropyl cellulose film, and administered to each Mongolian gerbil with 0.2 ml of water by using the polyethylene tube attached to a gastric sonde.

At 2 or 4 h after administration, the stomach of each animal is excised and the remaining amount of amoxicillin is evaluated, i.e., 40 ml of 1/15 M phosphate buffer (pH 7.2) is added to each stomach, and the amount of amoxicillin extracted is determined by a reversed-phase high-performance liquid chromatography (HPLC) method. The remaining percentage of amoxicillin as an index of residence in the stomach, i.e., mucoadhesiveness, is calculated by the following equation: remaining percentage=(amoxicillin remained/amoxicillin administered)·100. Correlation of the retained percentage versus initial dosage is plotted.

In addition, the concentration of amoxicillin in plasma is measured as follows. Amoxicillin is orally administered to 7-week-old male specific-pathogen-free Mongolian gerbils at a dose of 30 mg/kg in the form of amoxicillin-loaded silica-chitosan nanocomposite. HPLC is used to measure amoxicillin concentrations in blood samples (1 ml), collected by cardiac puncture at 1, 2, 4, or 6 h after administration while the gerbils are under ether anesthesia.

To investigate the in vivo antibacterial efficacy of the amoxicillin loaded nanocomposite, four-week-old male specific-pathogen-free Mongolian gerbils are fasted for about 24 h, and 1 ml of broth containing 107.63 CFU of H. pylori TN2GF4 per ml is inoculated into the stomach of each gerbil via an orogastric tube. Fourteen days after infection, amoxicillin is orally administered twice a day for three consecutive days at a dose of 1, 3, 10, or 30 mg/kg in the form of amoxicillin loaded chitosan-silica nanoporous composite. Empty silica-chitosan nanocomposite (with no drug) is administered as a placebo in the same manner. One day after administration of the final dose, the gerbils are sacrificed and the stomachs are removed. Each stomach is homogenized with brucella broth (3 ml/stomach), serial dilutions are plated on modified Skirrow's medium, then assayed for bacterial colony formation. In addition, gastric ulcer sites are collected from a separate experimental group and examined histological observation.

The above experiments demonstrate the effectiveness of the drug delivery system according to embodiments of the present invention.

In another embodiment of the present invention, recombinant human EGF (rhEGF) is loaded in the silica-chitosan nanocomposite to stably protect and enhance wound healing in the gastric mucosa. This embodiment demonstrated by utilizing the rhEGF-loaded silica-chitosan nanocomposite against ethanol-induced injury in rats.

SD rats weighing 200 to 250 g are used in the study of gastric protection and gastric ulcer. In brief, acute gastric lesion is induced by absolute ethanol in experiments with three sets of rats (control, rhEGF and rhEGF loaded chitosan-silica nanocomposite). The control group is given empty nanoporous composite (9 rats). The rhEGF group is given oral rhEGF 60 μg·kg-1·d-1 (9 rats). The rhEGF loaded chitosan-silica nanoporous composite is applied orally to another 9 rats. The rhEGF loaded chitosan-silica nanoporous composite is applied orally to another 9 rats. Three days later, 1 ml of absolute ethanol is administered to all rats. One hour after ethanol administration, the rats are sacrificed, the stomachs are dissected out and opened along the greater curvature, and the area of ulceration is determined. The amount of damage is expressed as ulcer index.

In addition, the measurement of serum EGF and gastrin level treated by rhEGF loaded nanocomposite are conducted as follows. Rat blood of 2 ml-4 ml is collected in a tube without an anticoagulant. Three hours later, the serum is collected and EGF and gastrin levels are measured. An EGF kit which is commercially available from Amersham, U.K., may be used for this procedure.

The surface area and pore volume of the dried nanoporous composites may be determined using, for example, a Quantasorb (Quantachrome Inc.) Brunauer-Emmett-Tellet (BET) analyzer using 30% N₂/He for single point analyses. Overall porosity may be determined from, for example, measurement of skeletal density using a helium pycnometer (Micromeritics AccuPyc 1330) and the bulk density (Micromeritics GeoPyc 1360 Envelope Density Analyzer). Infrared spectra may be recorded using, for example, a Nicolet Magna Fr-IR spectrometer from KBr pellets containing 1% of the powdered composites. The interaction between the silanol groups and the amido-carbonyl groups of the chitosan will be reflected in spectral shifts in the 1300-1750 cm⁻¹ region. Microscopy (TEM/SEM) on the dried nanocomposites may be taken using instrumentation which is commercially available, for example, from the Universities of Cincinnati and Maryland.

The following illustrate alternative embodiments for the preparation of silica-chitosan composites with encapsulated drugs.

A monolithic silica/chitosan composite is created using from about 1 to about 15, such as about 9 wt % silicic acid and from about 0.1 to about 3 wt %, such as about 2 wt % chitosan, solubilized in acetic acid solution sufficient to achieve an acidic pH needed for chitosan solubility (e.g., about 1% solution). Briefly, silicic acid may be generated from sodium silicate using an ion-exchange process as described previously. The low ionic strength of silicic acid allows for high loadings of hydrophobic drug molecules. Also, the acidic pH of silicic acid inhibits or prevents premature precipitation of chitosan prior to mixing. The chitosan composition of the composite may be easily varied by controlling the amount of chitosan solution introduced to a set volume of silicic acid.

### Monolithic Silica/Chitosan Composites

To make the monolithic silica/chitosan composite, the 2 wt % chitosan solution is weighed into a beaker. Then the silicic acid is added into the beaker and the contents stirred until homogenized. The solution is allowed to sit, without stirring, and gelation occurs within minutes; a silica gel with interpenetrating chitosan is formed.

### Drug Loading of Silica/Chitosan Composite

Drug loading may be achieved by, for example, any of the following methods: 1) mixing drug with chitosan solution prior to silicic acid addition; 2) mixing drug with silicic acid solution before addition to chitosan; and 3) adding drug after mixing of silicic acid and chitosan solutions. Slight differences in drug release rates may be seen depending on whether the drug is first added to chitosan or silicic acid.

### Effect of Drug Addition Sequence

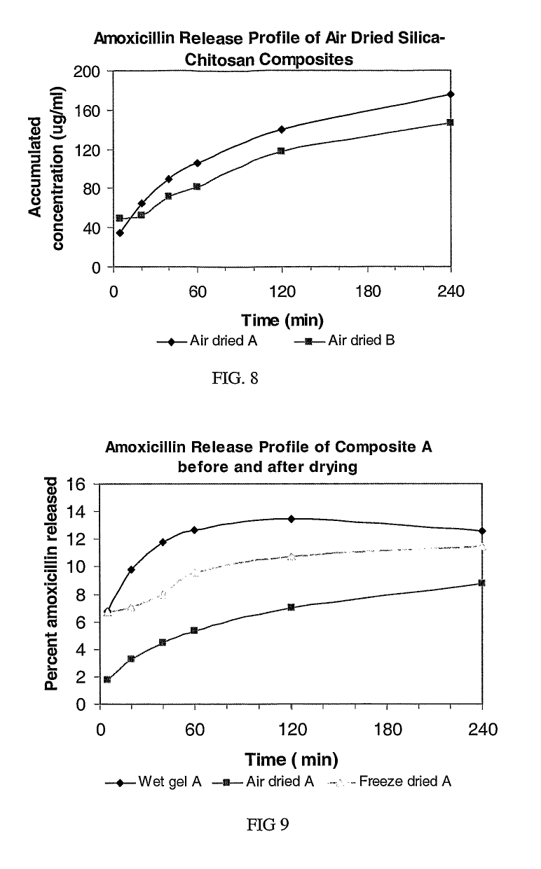

To determine the effect of drug addition sequence (i.e., to chitosan or silicic acid) on release rates, two silica chitosan composites are made with an identical chitosan/silica weight ratio of 0.11. These composites are also loaded with the same ratio of the antibacterial drug amoxicillin (AMOX)—AMOX/silica=5.6 (g/g) and AMOX/chitosan—1.0 (g/g). In composite A, amoxicillin is first added to chitosan before addition of silicic acid. In composite B, amoxicillin is first added to silicic acid before addition to chitosan. As shown in FIG. 8, the amoxicillin release rate slightly increases when first added to chitosan (composite A). Drug release studies are conducted with simulated gastric fluid at 37° C.

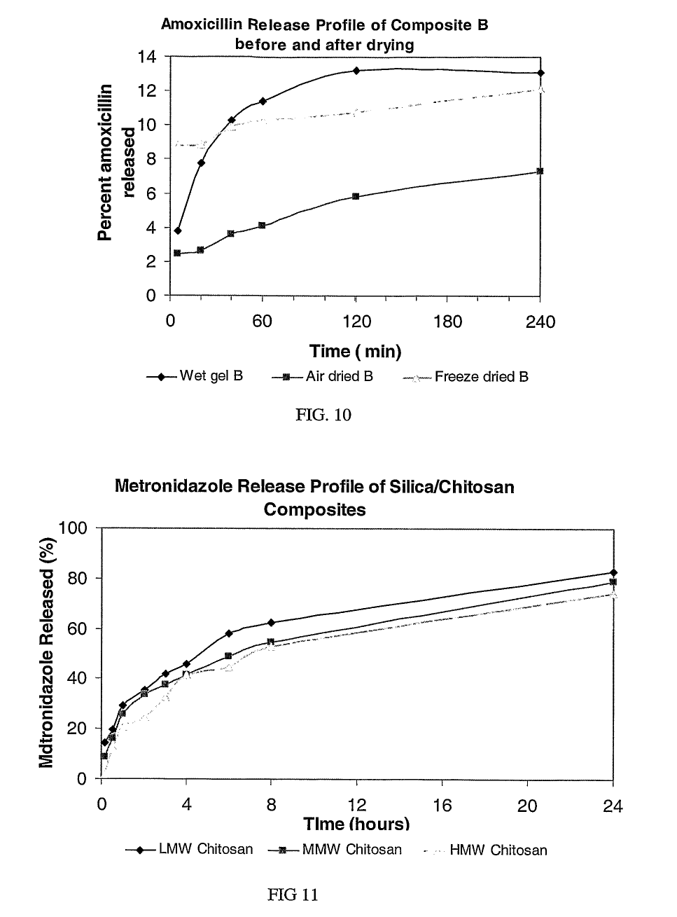

The effect of drying on the amoxicillin release profile is also determined. In brief, samples from composites A and B are dried by air or freeze drying. In addition, one set of sample from each composite are kept in a wet state to yield a total of six samples—2 wet (A & B), 2 air dried, and 2 freeze dried. As shown in FIGS. 9 and 10, the wet samples exhibit the fastest release rate in both composites A and B. The freeze dried samples are characterized with a large initial burst release of amoxicillin and then an apparently linear drug release profile. The air dried sample, which is expected to have the more dense morphology, show both a low burst and a slow release rate.

While these studies show the effect of drying on release rates of amoxicillin, they also indicate a fairly low stability of amoxicillin under the testing conditions. The wet gel profiles show that, while less than 14% of the entrapped drug is released after 2 hours, the amoxicillin measured in solution levels off. This is attributed to a low half-life of amoxicillin in low pH environments. See, Tapia-Albarran M, Villafuerte-Robles L. “Assay of amoxicillin sustained release from matrix tablets containing different proportions of Carbopol 971P NF” International Journal of Pharmaceutics 273:1-2 (2004) 121-127.

In order to evaluate the release profiles of the composites without the added complexity of drug degradation, metronidazole is used in subsequent release studies. Metronidazole possess greater stability under the testing conditions than amoxicillin. See, Wang D P, Yeh M K “Degradation Kinetics of Metronidazole in Solution” Journal of Pharmaceutical Sciences 82:1 (1993) 95-98.

As shown in FIG. 11, silica/chitosan composites loaded with Metronidazole show a total drug release of approximately 80 percent after 24 hours of incubation in simulated gastric fluid at 37° C. The composites shown in FIG. 11 contain a chitosan/silica weight ratio of 0.10 g/g. Metronidazole loading is 14.4 mg/g silica and 144 mg/g chitosan. While the chitosan/silica ratio is held constant, chitosan of varying molecular weights (i.e., low, medium, and high) are used in making the three different composites. The release profiles show that the performance of the composite is not greatly affected by the chitosan molecular weight. However, it appears that the initial burst size is decreased as chitosan molecular weight is increased.

The following examples illustrate embodiments of the invention for preparing substantially spherical silica/chitosan composites according to various embodiments of the invention, including, for example, an emulsion process and a precipitation process. The spherical composites generally allow for better control of the particle size and particle size distribution and may also provide more even distribution of drug throughout the composite and more uniform diffusion rates. Accordingly, in embodiments of the invention, the spherical silica/chitosan composites can provide for a more uniform (i.e., reduced variability) drug release and delivery rate, than the interpenetrating network structure.

### Spherical Silica/Chitosan Composites

### Emulsion Process

Using an emulsion process, as schematically illustrated in FIG. 12, spherical silica/chitosan composites are prepared. In this process, a chitosan solution is added to silicic acid and well mixed. This mixture is then added drop-wise to a stirred oil phase (e.g., 2-ethyl-1-hexanol), leading to formation of silicic acid/chitosan droplets in the bulk oil phase. Gelation of the silicic acid then results in the formation of silica spheres containing intertwined chitosan molecules. The composite particle size may be controlled, for example, by adjustment of the stirring rate and the addition of surfactants. Drug may be introduced to the silicic acid/chitosan solution, before addition to the oil phase, yielding silica/chitosan spheres with the entrapped drug. Depending on the solubility of the drug, one skilled in the art will be able to determine the best approach for adding the drug to the silica/chitosan composite. This method may be been used to fabricate silica/chitosan composites with a chitosan/silica weight ratio of up of about 20 percent, for example, from about 0.1 to about 20%, such as, at least about 0.5%, or 1%, or 2%, or 5% and up to about 20%, or 18% or 15% or 12% or 10% or 8%, or any intermediate or fractional value within these ranges.

### Precipitation Process

Spherical silica/chitosan composites may also be prepared using a precipitation/gelation process. For example, in one embodiment, a silicic acid solution, containing chitosan, is added drop-wise in a slowly stirred solution containing about 1% ammonia hydroxide. The basic conditions cause precipitation of chitosan, forming a shell, while silica gelation occurs. Droplets form with a sphere-like shape in the solution. Aging the droplets in the solution for about two hour yields the composite chitosan/silica spheres. The size of the composite spheres may be controlled, for example, by the tip of the device used to drop the silicic acid/chitosan solution. This method may be used to fabricate core-shell chitosan/silica composite with high chitosan/silica ratio of up to 80 percent.

As noted above, mucoadhesive drug delivery carriers, referred to as the Adhesive Micromatrix System, can adhere to the stomach wall in rats, thereby, remaining longer in the gastrointestinal (GI) tract. In recent years, chitosan and its derivatives have been widely assessed for the controlled release, or the delivery, of various drugs. Besides being biocompatible and biodegradable, chitosan offers advantages in drug delivery because of its permeation enhancement, mucoadhesiveness, and ability for sustained drug release. Illum et. al. reported that chitosan solutions, even at a low concentration (0.5%), are highly effective at increasing the adsorption of insulin across nasal mucosa in rats and sheep. It was suggested that the enhancement mechanism was a combination of bioadhesion and transient widening of the tight junction in a membrane.

However, due to its high solubility in acid, a composite form of chitosan is needed to prolong the residence and delivery time in the acidic environment of the stomach.

The following example demonstrates that the composite drug delivery system made by entrapping chitosan polymer within a silica network, in accordance with embodiment of the invention as described above, adheres to stomach mucosurface and delivers antibiotics closer to sites infected by H pylori.

In this example, in-situ gelation of surface modified silica networks in the presence of chitosan create an interpenetrating network of silica and chitosan macromolecules, such as shown in FIG. 1. Silica gel is very stable in acid. The tight entanglement structure in the silica-chitosan composite significantly retards chitosan's leaching under acidic environment. Therefore, in the gastric environment, this composite more effectively controls the drug release rate more effectively than bare chitosan.

In particular, this study compares the effect of the novel chitosan/silica nanocomposite compared with chitosan sponges on the mucosal adsorption of a model drug, amoxicillin, in vitro.

The chitosan-silical nanocomposite samples as shown in the following table are prepared:

| | Chitosan | Silica | Chitosan |
|---|---|---|---|
| Sample | (g) | (g) | (% wt) |
| A | 0 | 0.55 | 0 |
| B | 0.02 | 0.82 | 2.4 |
| C | 0.05 | 0.82 | 5.7 |
| D | 0.10 | 0.55 | 15.4 |
| E | 0.20 | 0.55 | 26.7 |
| F | 0.20 | 0.27 | 42.6 |

The dried nanocomposites (100 mg) were further treated with 50 mg of amoxicillin solubilized in PBS for 24 hrs, followed by freeze-drying. Drug content was measured by HPLC. A reverse-phase C18 column was used as stationary phase and trifluoroacetic acid 0.01M/methanol (80/20 v/v) at the flow rate of 1 ml/min as mobile phase. Mobile phase was monitored at the wavelength of 270 nm. Quantification of amoxicillin was conducted by using a calibration curve obtained using amoxicillin solutions at known concentrations.

### In Vitro Evaluation of the Mucoadhesive Properties of Chitosan-Silica Nanocomposite

Lyophilized chitosan-silica nanocomposites (samples with different compositions A-F) and chitosan were compressed into 5.0 mm diameter flat-faced discs. The compaction pressure was kept constant during the preparation of all discs. Tablets were attached with a pressure of 500 Pa to freshly excised intestinal rat mucosa, which has been fixed to a stainless steel cylinder (diameter 4.4 cm, height 5.1 cm, apparatus 4-cylinder, USP XXVI) using a cyanoacrylate adhesive. The cylinder was placed in the dissolution apparatus according to the USP, fully immersed with either 0.1 M HCl buffer (pH 2.0) or 100 mM phosphate buffer pH 7.4 at 37° C. and agitated with 125 rpm. The detachment, disintegration and/or erosion of test tablets were monitored over a 150 h time period.

### Drug Release Experiment

Sponges containing 10, 20, 40 mg of amoxicillin were compressed into tablets as described above. The release rate of amoxicillin from tablets was analyzed in vitro. Tablets were placed in a beaker containing 10 ml of 100 mM PBS buffer pH 7.4 at 37° C. Beakers were closed up and continuously shaken on an oscillating water bath. Aliquots were taken every hour and replaced with an equal volume of release medium equilibrated at 37° C. Sink conditions were maintained throughout the study. The amoxicillin concentration was determined using HPLC as described above.

### Cytotoxicity Test of Chitosan-Silica Nanocomposite

The NIH3T3 fibroblast cells were used for cytotoxicity testing by methyl thiazol-2-YL-2,5-diphenyl tetrazolium bromide (MTT) staining. After seven days, the cell-containing samples were rinsed with serum-free media to remove the unattached cells and were transferred to a new plate. Then, 250 μl MTT solution was added to each sample and incubated for 4 hours to induce MTT formazan formation. Purple formazan was extracted with dimethyl sulfoxide (DMSO), and was used for optical density (OD) measurement with a Thermomax ELISA reader at a wavelength of 540 nm with DMSO as a blank.

### Results

### Percentage of Drug Entrapment and Drug Release Study

Table 1 shows the effect of composition of chitosan-silica nanocomposite on drug entrapment efficiency. As the content of chitosan increased, the amount of entrapped drug decreased. This might be explained by increasing ionic repulsion between amoxicillin and chitosan, as chitosan content increase, as well as the more loose structure of nanocomposites with higher chitosan content.

TABLE 1
The drug content of chitosan-silica nanocomposite

| Type of composite | Drug content (%) |
|---|---|
| A | 80 ± 6 |
| B | 77 ± 4 |
| C | 78 ± 10 |
| D | 75 ± 8 |
| E | 60 ± 6 |
| F | 43 ± 13 |

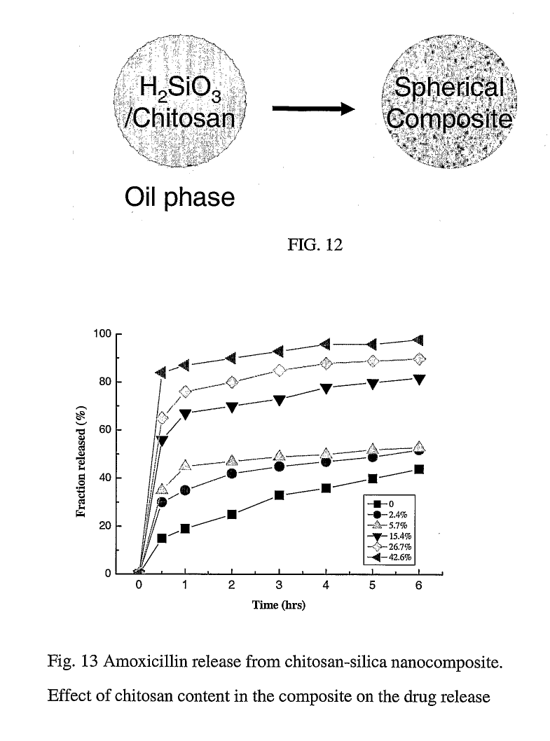

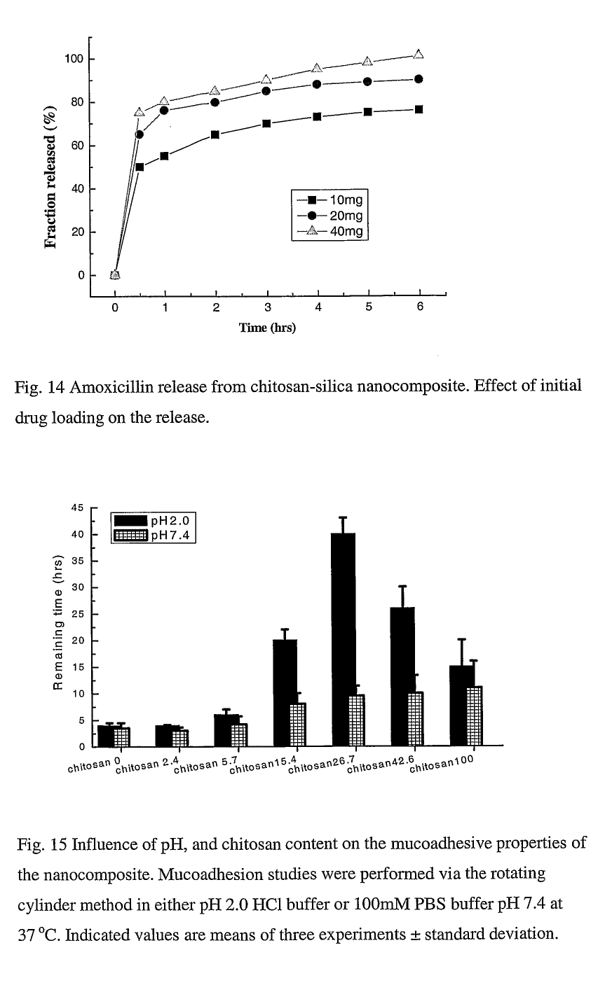

FIG. 13 demonstrates the effect the composition of chitosan-silica nanocomposites has on the release of amoxicillin. As seen from the profiles, drug release was retarded as the chitosan content decreases. As indicated above, composites with a higher chitosan content may be characterized with reduced rigidity, resulting in a fast wash-off of the entrapped amoxicillin.

The effect of initial drug loading on the drug release profile is shown in FIG. 14 (based on the E-form composite having a chitosan content of 26.7%). The release rate of amoxicillin remains high with high drug loading and decreases as the loading decreases. As a large fraction of the entrapped drug is exposed on the nanocomposite surface, an initial burst occurs at the beginning of the release experiments. The initial burst is advantageous, in the case of antimicrobial therapy, as the initial high dosage will provide strong bacterial suppression over a short period; this is followed by the inhibition of growth with the maintenance drug concentration.

### Mucoadhesiveness Studies

For the development of a strong mucoadhesive drug delivery system it was essential to optimize the adhesive properties of the chitosan-silica composite. Results shown in FIG. 15 demonstrate the mucoadhesive properties of chitosan-silica composites. The composite-mucosurface contact time was prolonged as the chitosan content increased up to 26.7 weight percent, indicating higher mucoadhesiveness. An increased chitosan content provides a higher proton concentration in the nanocomposite, thereby inducing significantly higher mucoadhesiveness when applied to the intestinal wall, abundant in sialic acid residue which can make hydrogen bonding with chitosan residue. However, at chitosan content of 42.6% and above the mucoadhesiveness is decreased, primarily due to the weak stability of the ionized network. The effect of pH on the mucoadhesiveness is also shown in FIG. 15. Under acidic conditions, chitosan was freely ionized and bound to the sialic acid in the intestine wall—providing higher mucoadhesiveness. This mucoadhesiveness is also governed by the structural integrity, as seen in FIG. 15; higher chitosan content reduced the adhesiveness. The composite showed almost similar mucoadhesiveness under pH 7.4, since the chitosan was intact at pH 7.4.

### Cytotoxicity Test

Table 2 demonstrates the cytotoxicity of chitosan-silica nanocomposite when applied to the culture of fibroblast cell line. As seen, all the tested samples did not possess any noticeable cytotoxicity, indicating the potential of safety when applied in the biomedical field.

TABLE 2
Viability of fibroblasts cultured with chitosan-silica composite.
Viability was determined as the ratio (%) of the absorbance
of sample group to that of group without treatment (NT)

| NT | | | | | | | Chitosan |
|---|---|---|---|---|---|---|---|
| (Control) | A | B | C | D | E | F | 100% |
| 100 ± 12 | 89 ± 10 | 88 ± 5 | 90 ± 4 | 93 ± 6 | 90 ± 5 | 87 ± 10 | 90 ± 9 |

What is claimed is:

1. A nanocomposite useful as a drug delivery system comprising chitosan polymer encapsulated in surface modified colloidal nanoporous nanoparticles.

2. A nanocomposite according to claim 1, wherein the colloidal nanoparticles comprise silica nanoparticles.

3. A nanocomposite according to claim 2, wherein the silica nanoparticles have a surface area of about 900 m²/g.

4. A nanocomposite according to claim 3, wherein the colloidal nanoparticles comprise surface ligand groups effective for interacting physically or chemically with a drug to be delivered.

5. A nanocomposite according to claim 4, wherein the nanoparticles comprise about 4 to about 5 ligand groups per square nanometer.

6. A nanocomposite according to claim 1, wherein the surface ligand groups comprise hydroxyl groups.

7. A drug delivery system comprising the nanocomposite according to claim 1 and a drug bound thereto.

8. A drug delivery system according to claim 7, wherein the drug comprises a hydrophilic drug.

9. A drug delivery system according to claim 7, wherein the drug comprises an antibiotic.

10. A drug delivery system according to claim 9, wherein the antibiotic comprises an antibiotic which is effective in the treatment of stomach ulcers.

11. A drug delivery system according to claim 9, wherein the antibiotic comprises amoxicillin.

12. A drug delivery system according to claim 9, wherein the antibiotic comprises tetracycline.

13. A drug delivery system according to claim 9, wherein the antibiotic comprises gentamycin.

14. A drug delivery system according to claim 7, wherein the drug comprises a hydrophobic drug.

15. A drug delivery system according to claim 7, wherein the drug comprises a polypeptide.

16. A drug delivery system according to claim 7, wherein the drug comprises epidermal growth factor.

17. A drug delivery system according to claim 7, wherein the composite comprises ligand groups inhibiting decomposition of drugs by an enzyme.

18. A drug delivery system according to claim 7, wherein the drug comprises a metal chelating agent.

19. A drug delivery system according to claim 18, wherein the chelating agent comprises an ethylenediamine effective for chelating zinc ions.

20. A drug delivery system according to claim 7, wherein the nanoparticles further comprises an oil phase within said nanopores.

21. A drug delivery system according to claim 20, wherein the drug is a hydrophobic drug which is compatible with said oil phase.

22. A method for treating stomach ulcers comprising administering to a patient in need of such treatment a mucoadhesive nanocomposite drug delivery system comprising a pharmacologically effective amount of a drug effective in the treatment of stomach ulcers embedded within a nanocomposite as set forth in claim 1.

23. A method according to claim 22, wherein the drug comprises an antibiotic.

24. A method according to claim 22, wherein the drug comprises an antibiotic effective against H. pylori.

25. A method according to claim 22, wherein the drug comprises amoxicillin.

26. A drug delivery system comprising a gelled silica-chitosan nanocomposite and a pharmacologically effective amount of a drug, wherein the drug is loaded into the nanocomposite by entrapment prior to gelation of the silica.

27. A method for preparing a nanocomposite useful as a drug delivery system comprising encapsulating chitosan polymer in surface modified colloidal nanoporous nanoparticles.

28. A method according to claim 27, which comprises dissolving chitosan polymer in an acidic, surface modified silica sol and increasing the pH to substantially neutral pH to thereby induce gelation.

29. A method according to claim 27, wherein the pH is raised to about 7.

30. A drug delivery system comprising an interpenetrating network of gelled silica comprising entrapped drug molecules and mucoadhesive chitosan biopolymer.

31. A nanodrug delivery system obtained by the method of claim 29.

32. A method for delivering a drug for extended release in the stomach of an animal in need thereof comprising administering to the animal the drug embedded in an acid-stable, nanocomposite material comprising an interpenetrating network of silica and mucoadhesive chitosan biopolymer.

33. A method according to claim 32, wherein the drug comprises an antibiotic effective against H. pylori.

34. A method according to claim 32, wherein the drug comprises amoxicillin.

35. A method according to claim 32, wherein the embedded drug is administered orally.

36. A method according to claim 32, wherein the embedded drug is administered via a nasal spray.

37. A method according to claim 32, wherein the embedded drug is administered via an implant.

38. A nanocomposite according to claim 1, wherein the nanocomposite is at least substantially spherical.

39. A nanocomposite according to claim 38, wherein the nanocomposite is prepared by an emulsion process.

40. A nanocomposite according to claim 38, wherein the nanocomposite is prepared by a precipitation/gelling process.

41. A drug delivery system according to claim 7, wherein the nanocomposite comprises at least substantially spherical particles.

42. A method according to claim 22, wherein the nanocomposite comprises at least substantially spherical particles.
